(19) United States
(12) Patent Application Publication
(10) Pub. No.: US 2002/0082493 A1
(43) Pub. Date: Jun. 27, 2002

(54) Precise and correct method of treating dystonia

(76) Inventor: Anuthep Benja-Athon, New York, NY (US)

Correspondence Address: ANUTHEP BENJA-ATHON, M.D., Rehabilitation Medicine, Acupuncture Physical Therapy, 210 East 36th Street, New York, NY, 10016, US

(21) Appl. No.: 09/748,302

(22) Filed: Dec. 27, 2000

(51) Int. Cl. A61B 5/05

(52) U.S. Cl. 600/410

(57) ABSTRACT

The discovery of the cause and mechanism of idiopathic torsion dystonia is described. A process of conveying the information on the causes and mechanism of the dystonia is also described. The method further comprises a process of identifying, testing, correctly treating the muscles producing and involving in dystonia and spastic and rigid muscles. Moreover, the method provides a precise process of rehabilitation including the use of noninvolved muscles, adaptive device and surgery to treat the dystonia.

### FIELD OF INVENTION

A precise and correct method of treating dystonia and spastic and rigid muscles.

### BACKGROUND OF THE INVENTION

The first objective of the present invention to affirm the discovery of the causes and mechanism of dystonia.

The second objective of the present invention is to identify and describe the causes and mechanism of said dystonia.

More specifically, it is the third objective of the present invention to claim the discovery of and to identify and describe the causes and mechanism of idiopathic torsion dystonia.

The fourth objective of the present invention is to provide a precise and correct method and approach to test and treat dystonia.

In a human, movement produce by muscles is an extremely complex neuromusculoskeletal process. It involves intricate neuronal organs, pathways and circuitry in the central and peripheral nervous systems. Said pathway and circuitry produce smooth, well-coordinated and purposeful movement of the body.

However, when there is a disruption in or a malfunction of said organs, pathways and circuitry, said smooth and purposeful movement are replaced by disruptive movement such as twisting and spasticity of agonistic and antagonistic muscle groups.

After Parkinsonism, dystonia and spastic and rigid muscles are common movement disorders afflicting humans. Dystonia can be devided into two categories based on the pathologic processes.

In the first category, it is labeled symptomatic torsion dystonia when the pathologic process is present. In this category, spastic and rigid muscles due to stroke and neurologic diseases are defined as and incorporated into this category of dystonia.

In the second category wherein the cause, pathogenesis and mechanism are not known, said dystonia is called idiopathic torsion dystonia or dystonia musculorum deformans. This category includes cervical dystonia or spasmodic torticollis or wry neck, blepharospasm including Meige syndrome, writer's cramp, spastic dysphonia and breathy dysphonia. (Merritt's Textbook of Neurology. Williams & Wilkins 1995)

Henceforth infra said two categories of dystonia will be referred to as dystonia.

In medicine, a disease wherein the pathogenesis or etiology is unknown, correct and precise treatment of said disease cannot be rendered. Said principle holds true for dystonia in which correct treatment and precise application of treatment are not available. Therefore, treatment of prior art is random involving the use of oral medications such as psychotrophic medications in the attempt, albeit unsuccessful, to control the dystonic movement. Neurotoxins such as botulinum toxins, phenol solution and alcohol are used in prior art to inject into to muscles which seemingly cause the dystonia. The result is weakness of the injected muscles and progressive weakness of the body part in the face of unabated dystonia. When the disease worsen, the dystonia becomes progressive, generalized and disabling. When all else failed, neurosurgery of prior art to severe the nerves to the muscles are performed. To say the least, the result is commonly disappointing to the physicians and therapists.

Even in symptomatic torsion dystonia, said dystonia including spastic and rigid muscle lack correct and precise treatment.

Consequently, most patients in their most productive years of life suffer pain, physical distortion, progressive disability and debilitation and emotional trauma. In some instances, mortality can be correlated said diseases.

In conclusion, said treatments of prior art are not beneficial to patients and often fail to contain or arrest the dystonia. This is due to the unknown pathogenesis or etiology of said dystonia. Moreover, rehabilitation and assistive device are not available since the causes and mechanism of dystonia are not known.

Through research of this physician applicant, the present invention claims the discovery of said causes and mechanism of said dystonia leading to proper and correct treatments including rehabilitation. The present invention also treats spastic and rigid muscles in patients suffered stroke and neurologic diseases.

### SUMMARY OF THE INVENTION

The cause, pathogenesis and mechanism of idiopathic torsion dystonia are not known. The correct and precise treatments of said dystonia are not available.

The present invention claims the discovery of the causes and mechanism of idiopathic torsion dystonia. Specifically said dystonia is caused by damage and injury to the muscle spindle comprising stretch receptors, intrafusal fibers and/or tendon organs of Golgi and their afferent and efferent nerves of a muscle or a group of muscles. Therefore, the origin of said dystonia is the damage, disordered and abnormal muscle spindle and/or tendon organ of Golgi and their innervating afferent and efferent nerves of a muscle or a group of muscles.

The present invention further provides a process of conveying said information on the causes and mechanism. The invention further comprises a process of identifying, testing, correctly treating the muscles producing and involving in dystonia and spastic and rigid muscles. Moreover, the method provides a precise process of rehabilitation including the use of noninvolved muscles, adaptive device and surgery to treat said dystonia.

### DESCRIPTION OF THE PREFERRED EMBODIMENTS

The present invention is the discovery of the causes and mechanism of dystonia. The present invention identifies and describes the causes and mechanism of dystonia. Specifically, the present invention is the discovery of, identification and description of the causes and mechanism of idiopathic torsion dystonia. Said dystonia includes, but not limited to, cervical dystonia or spasmodic torticollis or wry neck, blepharospasm including Meige syndrome, writer's cramp, spastic dysphonia and breathy dysphonia. Further to same, the present invention is an effective and correct method of treating said dystonia including spastic and rigid muscles as defined supra.

As a result of said achievement, the present invention includes the processes of describing, documenting, conveying and transferring the information on said causes and mechanisms using all means for communicating, describing, documenting, conveying and transferring of said information. Said information is communicated, described, documented, conveyed and transferred in print, copy and electronic means for communicating, describing, documenting, conveying and transferring of said information and the like.

The present invention is the discovery that said dystonia is caused by damage, injury to and abnormal function of the muscle spindle comprising stretch receptors, intrafusal fibers and innervating afferent and efferent nerves. As an extension, the present is also the discovery that said dystonia is caused by damaged, disordered and abnormal muscle spindles of a muscle or a group of muscles.

The present invention is the discovery that said dystonia is caused by damage, injury to and abnormal function of the tendon organ of Golgi and the innervating afferent and efferent nerves. As an extension, the present is also the discovery that said dystonia is caused by damaged, disordered and abnormal function of tendon organs of Golgi of a muscle or a group of muscles.

In other words, the origin of said dystonia is the damage and injury to muscle spindle comprising stretch receptors, intrafusal fibers and/or tendon organ of Golgi and their innervating afferent and efferent nerves. Said dystonia is caused by damaged, disordered and abnormal muscle spindles of a muscle or a group of muscles and/or damaged, disordered and abnormal tendon organs of Golgi of a muscle or a group of muscles.

The following represents a preferred embodiment of the present invention.

For example, it is the discovery affirmed in the present application that in writer's cramp wherein a writer repetitively wrote on contemporary triplicate documents, the cause of said dystonia in writer's cramp is the damage and injury to said muscle spindles, tendon organs of Golgi or combination thereof. In writer's cramp, through repetitive exertion of the flexor digitorum muscles in the repetitive process of said writing, the muscle spindles, tendon organs of Golgi or combination thereof of the flexor digitorum muscles of the affected upper extremity are damaged or injured. Said damage or injury is such as, but not limited to, sprain and strain, neuropraxia, neurotemesis, inflammation and disruption of said muscle spindles and/or tendon organs of Golgi.

In said instances wherein said muscle spindles and/or tendon organs of Golgi are damaged or injured, the corresponding muscle spindles and/or tendon organs of Golgi of the antagonistic muscle group to said agonist muscle or muscles are damaged or injured become unregulated and uncontrolled by the neuronal signal of said damaged or injured muscle spindles and/or tendon organs of Golgi. This interaction or event such as, but not limited to, reciprocal inhibition, occurred at the spinal cord and brain levels.

For example, in writer's cramp wherein said muscle spindles and/or tendon organs of Golgi muscles of flexor digitorum muscles of the affected upper extremity are damaged or injured. As a result of the improper or loss of input of signal from said damaged or injured muscle spindles and/or tendon organs of Golgi of ipsilateral flexor digitorum muscles, the muscle spindles and/or tendon organs of Golgi of ipsilateral extensor digitorum or wrist extensor muscles are not properly controlled or modulated at the spinal cord and brain levels. The output from the spinal cord and brain is the overactive and poorly modulated activity of said ipsilateral extensor digitorum or wrist extensor muscles.

In the attempt to correct or normalize the disorderly function of said flexors and extensors, the flexor groups such as, but not limited to, the ipsilateral wrist flexor muscle group is recruited. In addition, other ipsilateral proximal muscle groups may also be recruited such as, but not limited to, muscles of the upper arm and shoulder. These muscle groups will be called compensatory muscle groups (CMG). This recruitment is the attempt to control said extensor digitorum or wrist extensor muscles and to compensate for the imbalance or mismatch in the neural signal reaching the central nervous system. In other words, in the central nervous system such as, but not limited to, the spinal cord, there is interaction between the neurons of said nerves innervating said muscle spindles, tendon organs of Golgi and muscles. The outcome of said recruitment may or may not succeed in normalizing function of the writer's limb. The result is the torsional and twisting movement of said body part.

Kinesiology is used to identify the primary muscle group that contains the damaged or injured muscle spindle and/or tendon organ of Golgi. Similarly, the antagonistic muscles and CMG can be identified.

However, electrode means for determining said participation of agonistic, antagonistic muscles and CMG in said dystonia. For example, electromyographic activity of said muscles can be determined to establish the relationship of said muscle groups using surface electrode or electromyographic needle means for determining the myographic activities and contribution of said muscles to said dystonia.

Having said achievement, precise, accurate and correct treatment of the primary muscle groups initiating said dystonia and those muscle groups producing and involving in said dystonia can be undertaken. For example, in said writer's cramp the treatment involves the process of eliminating and preventing the cause which is the forceful and repetitive contraction of said flexor digitorum muscles in writing task. The process of healing comprises, but not limited to, rest and healing the inflammed and damged muscle spindles and tendon organs of Golgi. Medications such as oral medications, local anti-inflammatory and anesthetics can be used. In addition, correct and effective rehabilitation, physical therapy, biofeedback including visual, auditory and electromyographic feedback and acupuncture to reduce the neurogenic inflammation can be used.

Having said achievement, the process of precisely, specifically, correctly and accurately treating the muscles producing and involving in said dystonia using needle and needle-free means for percutaneously introducing chemicals, neurotoxins, local anti-inflammatory and anesthetics can be undertaken. As an example, individual member of the extensor muscle groups of the forearm in writer's cramp such as, but not limited to, extensor indicis propius, the extensor digitorum commnunis and/or wrist extensors can be injected. Therefore, the present invention provides correct, exact and precise targets for treatment.

Said needle-free means for percutaneously introducing chemicals, neurotoxins, local anti-inflammatory and anesthetics is a jet-injecting device. Said device does not engage a needle in the introduction of said chemicals, neurotoxins, local anti-inflammtory and anesthetics into said muscles. Said substances and solutions are propelled through the skin and into the target sites by a force produced by either compressed air, gas such as carbon dioxide, magnetics and mechanical means and the like. BIOJECT, Inc. produces needle-free injecting devices using pressure from compressed carbon dioxide contained in a cartridge. Said pressure propelled said substances and solutions at high speed through the skin and to said sites. Said achievement of the present invention provides this method of application of said needle-free means. Within said spirit of the present invention, the result is increased efficacy and less dose of said chemicals and drugs used with correct and accurate application of this method and the like.

Moreover, having said knowledge, electronic, electromyographic (EMG) and visual means for identifying, locating, defining and describing said target sites for said introduction of said chemicals, neurotoxins, local steroids and anesthetics is used. Said visual means is such as, but not limited, to the use of ultrasound, computerized tomography (CTScan) and magnetic resonance imaging (MRI) to visualize and localizing the precise, specific, correct and accurate sites of and within said muscles to be treated by said means.

In combination of all said procedures, treatments and processes, said electronic, electromyographic (EMG) and visual means are used in conjunction and in combination with said treatment means to optimize the treatment for said dystonia. For example, said visual means is used with needle and/or needle-free means for injecting said chemicals, neurotoxins, local anti-inflammtory and anesthetics into said muscles to obtain the most effective and accurate result of treatment.

For example, in writer's cramp EMG and MRI are used to localize all target sites such as, but not limited to, muscle spindles, tendon organs of Golgi, motor points, nerve branches and thickness of said muscles involved in said dystonia. Local anesthetics can be used as test drug to target selective sites in said muscles to be injected using said needle and/or needle-free (jet-injecting) injecting device means. The preliminary and reversible result is the determination of the outcome of said treatment. From said result, a more definitive, accurate and permanent result is obtained using said visual means, needle and/or needle-free (jet-injecting) injecting device means and said neurotoxins and the like such as, but not limited to, botulinum toxins, phenol solution and alcohol.

Having said achievement, the present invention also provides a precise process of rehabilitation of said dystonia comprising technique of isolating, suppressing and circumventing muscle groups causing and involving in said dystonia. The objective is to use different agonist and antagonist muscle groups and also other muscle groups adaptable to isolate, suppress and circumvent the neuronal circuitry that activates and perpetuates said dystonia.

For example, in the rehabilitation process of writer's cramp, visual, auditory and electromyographic means for identifying, isolating, suppressing and circumventing muscle groups causing and involving in said dystonia is used. In said case, it is the digital and wrist flexor and extensor groups which are identified, isolated, suppressed and circumvented. Muscles that are not involved in said dystonia can be recruited to produce normal or almost-normal function and movement of the affected limb. In this example, the interossei of the ipsilateral hand represents one set of said muscles.

Having said achievement, biofeedback means for isolating, suppressing and circumventing muscle groups causing and involving in said dystonia can be precisely and selectively use to accomplished supra. Said means also isolates, suppresses and circumvents the neuronal circuitry that produces and maintains said dystonia. Biofeedback means is such as, but not limited to, visual, auditory, tactile and electromyographic feedback for normal and sensory-input-deficit persons.

In addition, adaptive and rehabilitative device means for isolating, suppressing and circumventing said dystonia is used. The overall objective of said correlative device means is to avoid, circumvent and suppress said dystonia including the neuronal circuitry in producing and maintaining said dystonia. Said device means allows the affected body part to produce and execute normal or almost-normal function and movement.

For example, said adaptive and rehabilitative device for writer's cramp is a holder means for holding and fixing a position of a writing instrument in the first webspace of said writer using the interossei between the first and second digits of the affected limb. In a nutshell, said device is a firm rubber mass means for holding, gripping or fixing a pen or pencil between said first and second digits, i.e. thumb and index finger, respectively. Said device has the outer contour means for fitting the medial anatomy of said 1st and lateral anatomy of said 2nd digits. So designed, said device gives the most optimal biomechanical advantage for said interossei to optimally grips and fixing said device for writing. As a result, a dystonic upper limb can write with said device means using said digits and their interossei. In said setting, said device means not only isolates said flexor and extensor muscles causing and involving in said dystonia from the function and movement of the hand but also allows said hand to function and move normally in writing.

Surgical process of suppressing said dystonia is to correctly and exactly extirpate the nerves and the neuronal circuitry that cause and involve in said dystonia. For example, in writer's cramp, the target muscles are members of the flexor groups and/or the extensor groups in the forearm of the affected limb.

Moreover, surgical process can transplant and transpose correct and exact noninvolved muscle groups to take the place and function of said muscles causing and involving in said dystonia. For example, in writer's dystonia the members of the ipsilateral wrist flexors become candidates for transplantation and transposition.

The result of the present invention is the restoration of function and movement that fulfills the objectives of the present invention.

Corollary, said achievement, description, mechanism, applications and processes are applied to said dystonia such as, but not limited to, cervical dystonia, blepharospasm, spastic dysphonia and breathy dysphonia and also spastic and rigid muscles in patients who suffer stroke and neurologic diseases.

Although the preferred embodiments of the present invention have been described, it will be appreciated by those skilled in the art that adaptations and variations including means and methods may be made without departing from the spirit of the invention or the scope of the claims.

Although the preferred embodiments of the present invention including a device have been described, it will be appreciated by those skilled in the art that adaptations, variations and other device means for isolating and circumventing said dystonia may be made without departing from the spirit of the invention or the scope of the claims.

Although the present invention has been described for said movement disorders, it will be appreciated by those skilled in the art that said discovery, methods and processes can be applied to other diseases and illnesses without departing from the spirit of the invention or the scope of the claims.

Although this invention has been applied to human, it will be appreciated by those skilled in the art that its application and use can be applied in other living species without departing from the spirit of the invention or the scope of the claims.

Claims of the invention:

1. A precise and correct method of treating dystonia comprises:
a discovery of the causes of said dystonia;
a process of conveying the information on said causes of said dystonia;
a process of conveying the information on the mechanism of said dystonia;
a process of identifying the muscles producing and involving in said dystonia;
a process of testing the muscles producing and involving in said dystonia;
a process of precisely, specifically and accurately treating the muscles producing and involving in said dystonia;
a precise process of rehabilitation said dystonia;
a surgical process of suppressing said dystonia; and
a surgical process of transposing muscle means for circumventing said dystonia.

2. A precise and correct method of treating idiopathic torsion dystonia and spastic muscles comprises:
a discovery of the causes of said dystonia;
a process of conveying the information on said causes of said dystonia;
a process of conveying the information on the mechanism of said dystonia;
a process of identifying the muscles producing and involving in said dystonia;
a process of testing the muscles producing and involving in said dystonia;
a process of precisely, specifically and accurately treating the muscles producing and involving in said dystonia;
a precise process of rehabilitation said dystonia;
a surgical process of suppressing said dystonia; and
a surgical process of transposing muscle means for circumventing said dystonia.

3. The method according to claim 2 wherein said discovery is said dystonia is caused by damage, injury to and malfunction of the muscle spindle comprising stretch receptors, intrafusal fibers and innervating afferent and efferent nerves of a muscle group.

4. The method according to claim 2 wherein said discovery is said dystonia is caused by damaged and disordered said muscle spindle.

5. The method according to claim 2 wherein said discovery is said dystonia is caused by damage, injury to and malfunction of the tendon organ of Golgi and the innervating afferent and efferent nerves of a muscle group.

6. The method according to claim 2 wherein said discovery is said dystonia is caused by damaged and disordered said tendon organ of Golgi.

7. The method according to claim 2 wherein said process of conveying said causes of said dystonia is the description of a process of damage, injury to and malfunction of said muscle spindle.

8. The method according to claim 2 wherein said process of conveying said causes of said dystonia is the description of a process of damage, injury to and malfunction of said tendon organ of Golgi.

9. The method according to claim 2 wherein said process of conveying said causes of said dystonia is the description of the involvement of said damaged and disordered muscle spindle and tendon organ of Golgi and the innervating afferent and efferent nerves in said dystonia.

10. The method according to claim 2 wherein said process of conveying the information on the mechanism of said dystonia is the description of the neuronal circuitry in the peripheral and central nervous systems involving said muscle spindles, tendon organs of Golgi, agonistic and antagonistc muscle groups in producing said dystonia.

11. The method according to claim 2 wherein said process of conveying the information on the mechanism of said dystonia is the description of the interaction between muscle spindles, tendon organs of Golgi, agonistic and antagonistc muscle groups in producing said dystonia.

12. The method according to claim 2 wherein process of identifying the muscles producing and involving in said dystonia is the use of electromyographic and electrode means for determining the contribution of agonistic and antogonistic muscles in producing and involving in said dystonia.

13. The method according to claim 2 wherein process of identifying the muscles producing and involving in said dystonia is the use of auditory, tactile and visual means for identifying, locating, defining and describing muscles producing and involving in said dystonia.

14. The method according to claim 2 wherein said process of precisely, correctly and accurately treating the muscles producing and involving in said dystonia is the use of needle means for percutaneously delivering chemicals, neurotoxins, local anesthetics and anti-inflammatory medications to precisely and specifically deliver said chemicals, neurotoxins and local anesthetics to the muscles causing and involving in said dystonia.

15. The method according to claim 2 wherein said process of precisely, correctly and accurately treating the muscles producing and involving in said dystonia is the use of needle-free means for percutaneously delivering chemicals, neurotoxins, local anesthetics and anti-inflammatory medications to precisely and specifically deliver said chemicals, neurotoxins, local anesthetics and anti-inflammatory drugs to the muscles causing and involving in said dystonia.

16. The needle-free means according to claim 14 is a jet-injecting device means for percutaneously injecting said chemicals, neurotoxins, local anesthetics and anti-inflammatory drugs.

17. The method according to claim 2 wherein said precise process of rehabilitation said dystonia comprises the use of muscle group means for isolating, suppressing and circumventing said dystonia to produce normal or almost-normal function and movement.

18. The method according to claim 2 comprises a biofeedback means for isolating, suppressing and circumventing said dystonia to produce normal or almost-normal function and movement.

19. The method according to claim 2 wherein said precise process of rehabilitation said dystonia comprises the use of adaptive device means for isolating and circumventing said dystonia to to produce normal or almost-normal function and movement.

20. A method of treating idiopathic torsion dystonia and spastic muscle in a person in whom damaged, disordered and abnormal functioning muscle spindles and tendon organs of Golgi cause dystonia and spasticity of agonistic and antagonistic muscle groups whereby electronic, electromyographic and visual means for identifying, locating, defining and describing said muscle groups are used and whereby a precise, selective and correct application of needle and needle-free means for percutaneously delivering chemicals, neurotoxins, local anesthetics and anti-inflammatory drugs to said muscle groups comprises:
a discovery of the causes of said dystonia;
a process of conveying the information on said causes of said dystonia;
a process of conveying the information on the mechanism of said dystonia;
a process of identifying the muscles producing and involving in said dystonia;
a process of testing the muscles producing and involving in said dystonia;
a process of precisely, specifically and accurately treating the muscles producing and involving in said dystonia;
a precise process of rehabilitation said dystonia;
a surgical process of suppressing said dystonia; and
a surgical process of transposing muscle means for circumventing said dystonia.
